(19) United States
(12) Patent Application Publication
(10) Pub. No.: US 2009/0011407 A1
(43) Pub. Date: Jan. 8, 2009

(54) Polymorphic Cd24 Genotypes that are Predictive of Multiple Sclerosis Risk and Progression

(75) Inventors: Yang Liu, Ann Arbor, MI (US); Qunmin Zhou, Powell, OH (US); Pan Zheng, Columbus, OH (US); Kottil Rammohan, Columbus, OH (US); Shili Lin, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 10/596,062

(22) PCT Filed: Nov. 22, 2004
(86) PCT No.: PCT/US04/39391

§ 371 Date: Jun. 20, 2007

Related U.S. Application Data
(60) Provisional application No. 60/525,502, filed on Nov. 26, 2003.

(51) Int. Cl. C12Q 1/68; C12Q 1/02

(52) U.S. Cl. 435/6; 435/29

(57) ABSTRACT

An image data correction apparatus has a motion information acquisition section, a correction section, and a composition section. The motion information acquisition section acquires motion information indicating spatial distribution of the magnitude of motion, in actual space, of a to-be-imaged portion of a subject. Based on the motion information, the correction section performs correction, which is different from correction in a second region, in a first region of image data collected by a scan by magnetic resonance imaging. The composition section composes individual image data of the first region and the second region that are corrected by the correction section.

This application claims priority to U.S. Provisional Patent Application 60/525,502, filed Nov. 26, 2003.

Research leading to this invention was supported, at least in part, by NCI Grant No. CA90223. The Federal Government has certain rights in this invention.

### FIELD OF THE INVENTION

The invention relates to genetic analysis of CD24 gene for predicting risk and progression of multiple sclerosis and for designing differential treatment of multiple sclerosis depending on the allotype of the CD24 gene.

### BACKGROUND OF THE INVENTION

Multiple sclerosis (MS) is a chronic inflammatory disorder in the central nervous system (CNS) that affects approximately 0.1% of Caucasians of Northern European origin (1) (approximately 250,000 individuals in the United States). The incidence of MS is increased among family members of affected individuals. The concordance rate of identical twins can be as high as 30% (1) (2, 3). Although the clinical course may be quite variable, the most common form of MS is manifested by relapsing neurological deficits, in particular, paralysis, sensory deficits, and visual problems. The inflammatory process occurs primarily within the white matter of the central nervous system and is mediated by T lymphocytes, B lymphocytes, and macrophages. These cells are responsible for the demyelination of axons. The characteristic lesion in MS is called a plaque. Multiple sclerosis is thought to arise from pathogenic T cells that somehow evaded mechanisms establishing self-tolerance, and attack normal tissue. T cell reactivity to myelin basic protein may be a critical component in the development of MS.

An individual with clinically definite MS has had two attacks and has presented with clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. Slightly lower criteria are used for a diagnosis of clinically probable MS. Clinical progression of multiple sclerosis may be examined in several different ways. Three main criteria are used: EDSS (extended disability status scale), appearance of exacerbations, or MRI (magnetic resonance imaging).

The EDSS is a means to grade clinical impairment due to MS (Kurtzke, Neurology 33:1444, 1983). Eight functional systems are evaluated for the type and severity of neurologic impairment. Prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). A decrease of one full step defines an effective treatment in the context of the present invention (Kurtzke, Ann. Neurol. 36:573-79, 1994).

MRI can be used to measure active lesions using gadolinium-DTPA-enhanced imaging (McDonald et al. Ann. Neurol. 36:14, 1994) or the location and extent of lesions using T₂-weighted techniques. Baseline MRIs are obtained. The same imaging plane and patient position are used for each subsequent study. Positioning and imaging sequences are chosen to maximize lesion detection and facilitate lesion tracing. The same positioning and imaging sequences are used on subsequent studies. The presence, location and extent of MS lesions are determined by radiologists. Areas of lesions are outlined and summed slice by slice for total lesion area. Three analyses may be done: evidence of new lesions, rate of appearance of active lesions, and percentage change in lesion area (Paty et al., Neurology 43:665, 1993).

No curative treatment for MS has been established. Corticosteroids and ACTH have been used to treat MS. Basically, these drugs reduce the inflammatory response by toxicity to lymphocytes. Recovery may be hastened from acute exacerbations, but these drugs do not prevent future attacks or prevent development of additional disabilities or chronic progression of MS (Carter and Rodriguez, Mayo Clinic Proc. 64:664, 1989; Weiner and Hafler, Ann. Neurol, 23:211,1988). Other toxic compounds, such as azathioprine, a purine antagonist, cyclophosphamide, and cyclosporine have been used to treat symptoms of MS. As with corticosteroid treatment, these drugs are beneficial at most for a short term and are highly toxic. Side effects include increased malignancies, leukopenias, toxic hepatitis, gastrointestinal problems, hypertension, and nephrotoxicity (Mitchell, Cont. Clin. Neurol. 77:231, 1993; Weiner and Hafler, supra). Antibody-based therapies directed toward T cells, such as anti-CD4 antibodies, and anti-CD24 antibodies may also be useful, though these agents may cause deleterious side effects by immunocompromising the patient. Several forms of beta interferon have been approved for use in MS patients.

The HLA locus is perhaps an important genetic element for MS susceptibility, as the HLA-DR2 allele has been identified as an important susceptibility gene among Caucasians (4-10). A majority of MS patients have HLA-type DR2a and DR2b. In addition, several additional loci have been proposed (8-12). Whole genome scanning has suggested a linkage-disequilibrium in the distal region of chromosome 6q (8), whose identity has not been revealed. An interesting candidate in the region is CD24 (13). We have previously shown that expression of CD24 is essential for the induction of experimental autoimmune encephalomyelitis (EAE) in mice (13).

CD24 is a glycosylphosphatidyl-inositol (GPI)-anchored cell surface protein with expression in a variety of cell types that can participate in the pathogenesis of MS, including activated T cells (14, 15), B cells (16), macrophages (17), dendritic cells (18), and local antigen-presenting cells in the CNS, such as vascular endothelial cells, astrocytes, and microglia (our unpublished observation). It is well established that in the mouse CD24 mediates a CD28-independent co-stimulatory pathway that promotes activation of CD4 and CD8 T cells (16-21). In addition, CD24 has been shown to modulate the VLA4-fibronectinNCAM-1 interaction (22), which is required for the migration of T cells to the CNS, and therefore the development of EAE in the mouse (23). We have recently demonstrated that CD24 is required for the development of EAE in the mouse (13). Interestingly, CD24 controls a checkpoint of EAE pathogenesis after the autoreactive T cells are produced (13).

Despite what is known about MS, the methods available to predict an individual's likelihood of developing MS remain inadequate. Likewise, no generally accepted methods are available to predict the aggressiveness of MS in patients that have been diagnosed with the disease. Accordingly, it would be desirable to have methods for screening the genetic profiles of individuals who are at risk for MS or known to have MS so as to better predict the development and course of disease in such individuals, and to customize treatment based on an individual's genetic profile.

### SUMMARY OF THE INVENTION

As described herein, it has been discovered that the presence of a single-nucleotide polymorphism (SNP) in the human CD24 gene is correlated with risk for developing MS, and with the rate of progression of the disease in patients diagnosed with MS. In particular, it has been discovered that the presence of a SNP within the nucleotide sequence encoding the CD24 gene product is positively correlated with increased incidence and more rapid progression of MS in a sample population assessed as described herein. As used herein in reference to MS, the term “rapid progression” means that an individual has reached or will reach EDSS 6.0 in a shorter time period than average from the time of first diagnosis of MS.

In one embodiment, a single nucleotide polymorphism from C (cytosine) to T (thymidine) at nucleotide position 226 in exon 2 of the coding sequence of the CD24 gene, resulting in an amino acid change from A (alanine) to V (valine) at amino acid position −1 (relative to the cleavage site of the mature, membrane-inserted protein), is positively correlated with an increased risk for developing MS and with more rapid progression of MS in the sample population assessed as described herein. The wild-type allele at position 226 is designated herein as “CD24^{226a}” and the variant allele is designated herein as “CD24^{226v}”, This particular polymorphism may be one of a group of two or more polymorphisms in the CD24 gene, or linked genes, which contributes to the development and progression of MS. As used herein in connection with the nucleotide at position 226 and the corresponding amino acid in CD24, the term “wild-type” refers to the allele for alanine and the term “variant” refers to an allele that differs or varies from the wild-type allele, such as the allele for valine which is described herein. Use of the terms wild-type and variant is merely for convention, and is not intended to suggest that either allelic form is a mutant of the other.

A wild-type or variant allele, such as either CD24^{226a} or CD24^{226v}, can be detected by any of a variety of available techniques, including: 1) performing a hybridization reaction between a nucleic acid sample and a probe that is capable of hybridizing to the allele; 2) sequencing at least a portion of the allele; or 3) determining the electrophoretic mobility of the allele or fragments thereof (e.g., fragments are generated by endonuclease digestion, then analyzed by a technique such as RFLP). The allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g., RT-PCR and allele specific amplification). Oligonucleotide primers that are directed to target sequences upstream and downstream of nucleotide position 226 and necessary for amplification may be selected for example, from within the CD24 gene, either flanking the SNP location, for example nucleotide position 226 (as required for PCR amplification), or directly overlapping the SNP location, for example nucleotide position 226 (as in ASO hybridization). In a particularly preferred embodiment, the sample is hybridized with a set of primers, which hybridize 5′ and 3′ in a sense or antisense sequence to the SNP, and is subjected to a PCR amplification.

An allele may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications. In a particularly preferred embodiment, the level of expression of the protein is evaluated based on the presence of the protein on the surface of cells, preferably peripheral blood lymphocytes, and most preferably T cells.

In one embodiment, the invention relates to a method for predicting the likelihood that an individual will have or develop MS, or that an individual who has been diagnosed with MS will experience more rapid progression of the disease, comprising the steps of obtaining a polynucleotide sample from an individual to be assessed and determining the nucleotide present at nucleotide position 226 of the CD24 gene. The presence of a “TV” (the variant nucleotide) at position 226 indicates that the individual has a greater likelihood of having MS than an individual having a “C” at that position. The presence of a “T” (the variant nucleotide) at position 226 in both alleles (i.e., homozygous for the CD24^{v} allele) indicates that an individual who has been diagnosed with MS has a greater likelihood of experiencing more rapid progression of MS as compared to individuals who are either homozygous for the wild-type CD24^{a} allele or are heterozygous (CD24^{a/v}).

In another embodiment, the invention relates to a method for diagnosing an individual as having or likely to develop MS, or of predicting that an individual who has been diagnosed with MS will experience more rapid progression of the disease, comprising the steps of obtaining a nucleic acid sample from an individual to be assessed, determining the HLA genotype of the individual, and determining the nucleotide present at nucleotide position 226 of the CD24 gene. The presence of the HLA genotype DR2 together with the presence of a “T” (the variant nucleotide) at both alleles of position 226 (i.e., homozygous for the CD24^{v} allele) indicates that the individual has a greater likelihood of having MS than an individual lacking the DR2 genotype and having a “C” at position 226, and that an individual who has been diagnosed with MS has a greater likelihood of experiencing more rapid progression of MS as compared to individuals who are either homozygous for the wild-type CD24^{a} allele or are heterozygous (CD24^{a/v}).

In yet another embodiment, the invention relates to a method for predicting the likelihood that an individual will have or develop MS, or that an individual who has been diagnosed with MS will experience more rapid progression of the disease, by determining the level of cell-surface expression of CD24 in the individual. The method comprises obtaining a cell sample from an individual to be assessed, wherein the sample comprises cells, preferably peripheral blood lymphocytes, most preferably T cells, wherein CD24 is expressed on the cells surfaces thereof. The level of cell-surface expression of CD24 is determined, wherein an increased level of expression as compared with control cells correlates with the presence of a SNP at nucleic acid position 226 in the CD24 gene, and indicates that the individual has an increased likelihood of developing MS. In one embodiment, the level of cell surface expression of CD24 is determined by contacting the cell sample with an excess of fluorochrome-labeled anti-human antibodies specific for CD24 in conjunction with antibodies specific for CD3 (T-cell markers), and determining the level of binding of the antibodies on a per-T cell basis using flow cytometry.

The invention is also drawn to kits for use in the methods of the present invention. In one embodiment, the kit comprises a nucleic acid probe, wherein said probe allows the identification of the nucleotide at position 226 of the CD24 gene. The kit can also include control nucleic acid samples. The control nucleic acid samples can include, for example, the homozygous wild-type genotype, homozygous variant genotype and the heterozygous genotype at nucleotide position 226 of the CD24 gene. In one embodiment the kit comprises control nucleic acid samples representing the genotype of at least one of the group consisting of: an individual homozygous for a “T” at nucleotide position 226 of a CD24 gene, an individual homozygous for a “C” at nucleotide position 226 of a CD24 gene and an individual heterozygous for said position.

In another embodiment, the kit comprises at least one antibody, selected from the group consisting of: an antibody specific for CD24 or fragment thereof and an antibody specific for T cells.

The inventive methods are advantageous in that they provide predictive information regarding the risk that an individual will develop MS and the likelihood that an individual who has been diagnosed with MS will experience rapid progression of the disease. Such predictive information can be used to assist in further evaluation of an individual to determine whether they have or may develop MS. Such predictive information may also be used to develop customized treatment plans for the individual. The design of such customized plans may involve altering the timing and dosage of standard treatment regimens based on whether the individual is heterozygous for the variant allele or homozygous for either the wild-type or variant allele at position 226. By customizing treatment of MS based on a patient's CD24 genetic profile, an improved outcome may be achieved for the patient, along with time and cost savings that are afforded by foregoing unnecessary therapy.

### BRIEF DESCRIPTION OF THE DRAWINGS

FIG. 1 shows the distribution of CD24 genotypes among MS patients and normal population control. a. The reported SNP of CD24 gene and its resulted amino acid replacement. Note that the Alanine (A) to Valine (V) change occurs immediately preceding the site (ω) for the GPI cleavage. b. Example of genotyping by PCR followed by restriction enzyme digestion. The samples are from normal donors. The genotypes of the individuals are marked in the lanes. c. Distribution of CD24 genotypes among normal population control (unfilled bars), and MS patients (filled bars). The data are based on analysis of 207 normal control and 242 MS patients. The distribution of the genotypes is as follows: normal (CD24^{a/a}:109, CD24^{a/v}:85, CD24^{v/v}:13) and MS (CD24^{a/a}:113, CD24^{a/v}:97, CD24^{v/v}132). The p values are given in the panel.

FIG. 2 shows MS types of MS patients for whom CD24 genotype analyses were conducted. The diagrams of type I(a) and type II(b) families used for the TDT analysis. The numbers in the parentheses following the genotypes are the ages of the donor when the samples were collected. For patients with genetic data, the EDSS scores were also provided. The nuclear families used for analysis are circled.

FIG. 3 shows CD24 genotypes and the time-span of MS patients from the year of first MS symptoms to the year they reached EDSS 6.0. Note that 50% of patients with CD24^{v/v} genotype reached EDSS 6.0 by 5 years as compared to 13 years for the CD24^{a/a} or 16 years for CD24^{a/v} patients. The p values are given in the panel.

FIG. 4 shows results of peripheral blood lymphocyte analyses comparing expression levels of various CD24 alleles. Higher expression of CD24 on T cells from patients with CD24^{v} allele. PBL was isolated from blood of 10 MS patients who belong to either CD24^{a/a} or CD24^{v/v} genotypes with approximate match in age, sex and EDSS (see Table 1 for details). The cells were stained for CD3 and CD24 markers. a. Contour graphs depicting expressing of CD24 and CD3 among the PBL of a representative patient in CD24^{a/a} and CD24^{v/v} groups. b. The mean fluorescence of total PBL or gated CD3⁺ T cells. Data presented are means and SEM (n=5). c, as in b, except that the expression of CD24 was compared between CD24^{a/a} and CD24^{a/v} patients (n=6).

FIG. 5 shows results of in vitro experiments comparing expression levels of various CD24 alleles. CD24^{v} is expressed at higher levels than CD24^{a} allele in both transient (a) and stable (b) CHO cell transfectants. CD24^{v} and CD24^{a} were cloned into PCDNA3 vector. a. CHO cells were transfected with varying amounts of CD24 cDNA. At 65 hours after transfection, the transfected CHO cells were stained with saturating amounts of PE-conjugated anti-CD24 mAbs. The y-axis, the CD24 expression, shows the products of % of CD24 expressing cells and mean fluorescence intensity of the positive cells. The means+/−S.D. of triplicate samples are shown. The data are representative of 3 independent experiments. b. Comparison of CD24^{v} and CD24^{a} expression after removing non-expressing cells by neomycin selection. At 48 hours after transfection, the CHO cells were selected with G418. The short-term drug-resistant culture (consisting of about 500-1000 clones) were pooled and stained with saturating amounts of PE-conjugated anti-CD24 mAbs. Data shown were means±S.D. of three independent analyses. The background fluorescence of untransfected CHO cells was subtracted. The p values from student t-tests are given in the panels.

FIG. 6 shows CD24 genotypes at P1580 and progression of multiple sclerosis. See FIG. 3 legends for detail.

FIG. 7 shows the polynucleotide sequence for human CD24.

FIG. 8 shows the polypeptide sequence for human CD24.

### DESCRIPTION OF THE EMBODIMENTS

Much of the genetic variation between organisms of the same species is a result of random mutation at specific nucleotide positions which results in the creation of multiple allelic forms of the same gene. As used herein, polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair, in which case it is referred to as a single nucleotide polymorphism. These single nucleotide polymorphisms (SNPs, pronounced snips) have the potential to produce profound effects on gene expression and consequently phenotype. For example, a SNP can alter the stability of mRNA by changing binding sites or secondary structure, thus making the mRNA more or less likely to be degraded. A SNP can change promoter binding sites and thereby modify the affinity for a transcription factor. Nonsense SNPs can introduce a premature stop codon that produces a truncated polypeptide, often resulting in loss of function of the gene product. Missense SNPs result in amino acid changes that can result in a functional change in the gene product if the properties of the new amino acid (charge, polarity, etc) are different from the one it replaced.

We have previously reported a critical role for CD24 in the development of EAE (13), the mouse model for MS. To explore the significance of this finding in human MS, we addressed the potential contribution of polymorphisms in MS susceptibility. It has been described that the human CD24 gene has a SNP that encodes a non-conservative replacement of an amino acid (from Alanine in CD24^{226a} to Valine in CD24^{226v}) immediately preceding the putative cleavage site for the GPI anchor (ω-1 position) (24). Here we show that the CD24^{226v/v}, genotype is associated with increased risk for developing MS and more rapid progression of MS in patients diagnosed with the disease. As we describe herein, the CD24^{226v} is more efficiently expressed on the surface of T lymphocytes, and other cells, in contrast to CD24^{226a}. This effect on cell surface expression may influence MS pathogenesis. To our knowledge, this is the first SNP to have a significant impact on MS susceptibility and disease progression. Since MS patients have high frequency of autoreactive T cells, molecules that control events after T cell activation present unique therapeutic targets. CD24 is one such post-T cell activation target for therapy of human MS. Our data reported here provide three lines of evidence for a significant contribution of the CD24 polymorphism at nucleic acid position 226 to the risk and progression of MS.

First, analysis of the distribution of the CD24 genotypes among more than 200 MS patients and the general population of the central Ohio region indicated that the frequency of the CD24^{226v/v} genotype in MS patients is more than twice that of the general population. This result suggests the CD24^{226v/v} homozygocity raises the relative risk of MS by more than 2-fold. It would be of great interest to test this correlation in other cohorts.

Second, using the combined TDT and S-TDT tests, we showed that the CD24^{226v} allele is preferentially transmitted to the affected individuals in comparison to unaffected individuals. These data confirm that the association at the population level most likely reflects that either CD24 or a gene linked to CD24 contributes to MS susceptibility in human.

Third, in addition to an increased risk of MS, the MS patients with CD24^{226v/v} genotype also have a more rapid progression, as judged by the time lapse between the first MS symptom and the time when a walking aid needs to be prescribed. We have chosen EDSS 6.0 as the pre-determined endpoint in experimental designs as this is a readily identifiable milestone in MS progression. We found that among the patients that have reached EDSS 6.0, 50% of the CD24^{225v/v} patients reached that milestone in 5 years, while CD24^{226a/a} and CD24^{226a/v} patients did so in 13 and 16 years, respectively. More rapid progression in the CD24^{226v/v} patients suggests that more aggressive treatment may be warranted in this group of patients.

An important issue is how the CD24 SNP at nucleic acid position 226 affects the risk and progression of MS. The CD24 gene product is a GPI anchored molecule with approximately 32 amino acids in the mature protein (after post-translational cleavage of portions). The SNP at nucleic acid position 226 in CD24 results in a non-conservative replacement from Alanine to Valine at the site immediately preceding the putative cleavage site for GPI anchor (called the ω-1). Although strict conservation at this site is not necessary for the cleavage and anchor attachment, there appears to be a general requirement for the total sites of the 4 amino acids at positions ω+1, +2 ω-1, and −2 (34). Since the Alanine and Valine have a substantial difference in size, it is plausible that these two alleles may be expressed at slightly different efficiency. Our comparison revealed that the CD24^{226v} allele is expressed at 30-40% higher levels than the CD24^{226a} allele.

Indeed, the T cells in the peripheral blood of the CD24^{226a/v} patients expressed significantly higher levels of CD24 than those in the blood of the CD24^{226a/a} patients. Although resting T cells expressed very little CD24 in the mouse, its expression is rapidly induced after activation (14, 23). Since our previous work established that CD24 gene must be functional in T cells for the T cells to be pathogenic (13), the induction of CD24 in T cells may be an important checkpoint for the pathogenesis of MS. For this reason, more efficient expression of CD24^{226v} alleles on T cells may provide a plausible explanation for the increased risk and progression of MS in the CD24^{226v/v} patients. The more efficient expression of CD24, however, is not necessarily limited to T cells, as the CD24^{226v} cDNA is more efficiently expressed even in CHO cells. Thus, the statistically insignificant difference among total PBL is most likely secondary to the vast variation in the proportion of leukocyte subsets with varying levels of CD24 (data not shown).

CD24^{226v/v} Genotype and Increased MS Risk in Population Study

We obtained 207 unused blood samples from the American Red Cross in Columbus and 243 samples of MS patients for the distribution of CD24 genotypes. The demography of the normal control population was not collected among the American Red Cross samples, but is assumed to reflect the general demography of the Central Ohio population. Moreover, the distribution of the CD24 genotype among our control population is similar to what was reported in a small population analysis in Europe (24). Among the 242 MS samples, 233 were from Caucasian, 7 were from African-American, 1 from Hispanics and one from Asian. The race distribution of the samples reflected both the demography of the Central Ohio population and the higher incidence of MS among the Caucasian, but not selective recruitment.

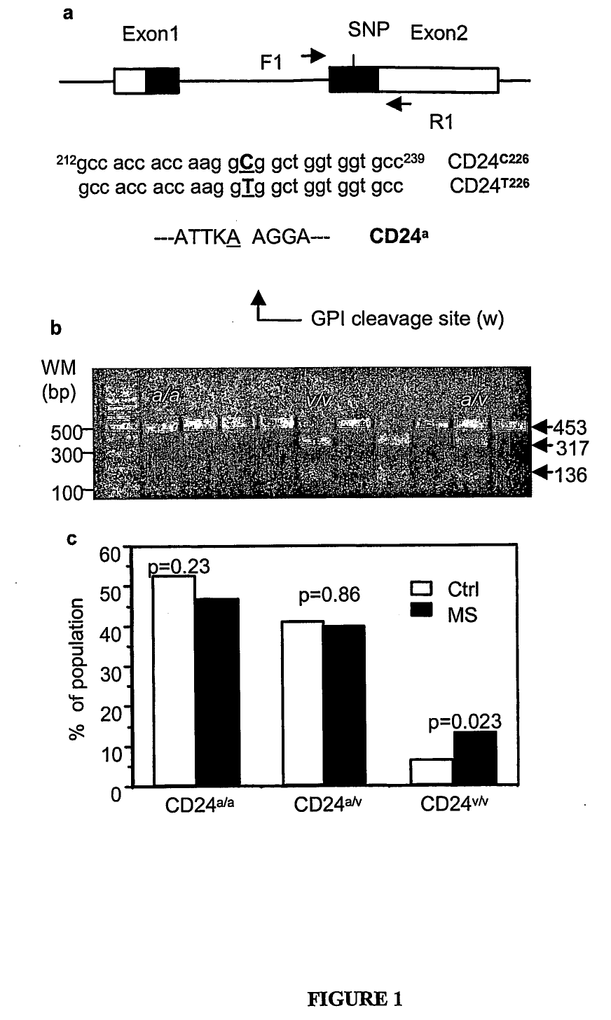

As shown in FIG. 1a, the CD24 genotype can be distinguished by digesting the PCR products of CD24 with BstXI. The CD24^{226a/a} products were completely resistant to the digestion, while the CD24^{226v/v} products cleaved into two fragments of 317 and 136 bp. Partial digestion of 50% or less indicated CD24^{226a/v} genotype. We therefore used this method to genotype the DNA isolated from leukocytes of normal population control and MS patients. The distribution of the genotypes among normal (CD24^{226a/a}:109, CD24^{226a/v}:85, CD24^{226v/v}:13) and MS (CD24^{226a/a}:113, CD24^{226a/v}:97, CD24^{226v/v}32) were compared by the Chi-square test. It was revealed that the distribution of CD24 genotypes among the MS patients appeared to differ significantly from that of the normal controls (p=0.048). The difference is significant among the CD24^{226v/v} genotype (6.3% in control vs 13.2% in MS, p=0.023), even after Bonferroni correction for multiple testing. The increased risk among the CD24^{226v/v} individuals of about 2-fold suggests that the CD24 gene may be a modifier for MS susceptibility. Although some of the patients are related, they are treated as independent samples in the tests.

Association of the CD24^{226v} Allele with MS in Family Study

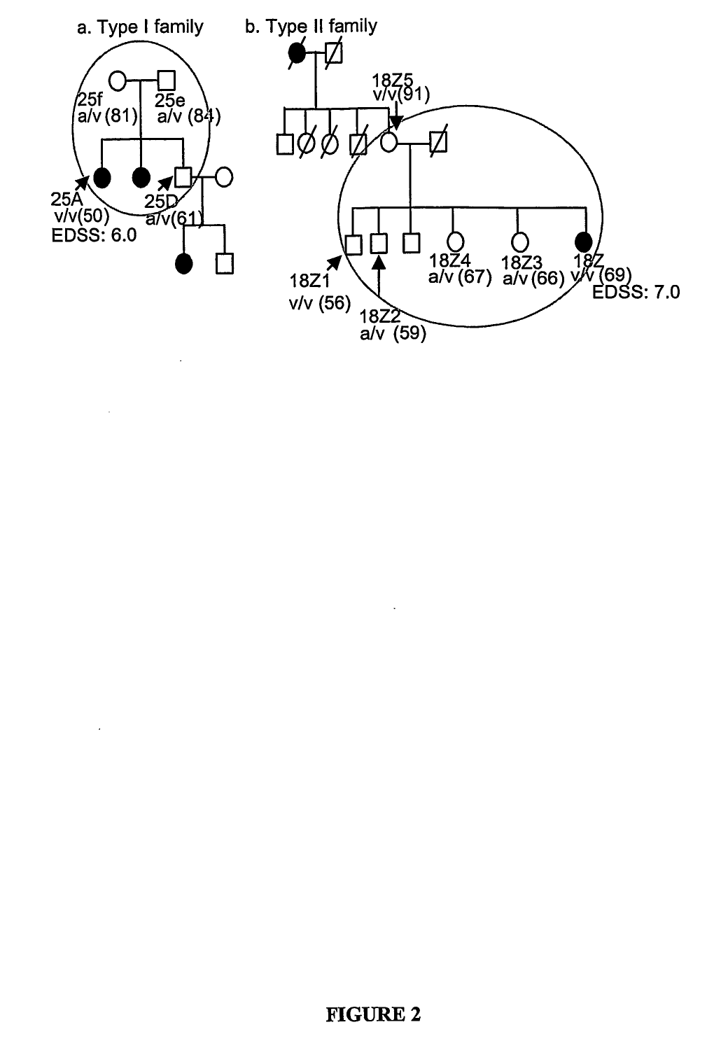

Eleven trios (type I families) and 18 sibships (type II families) from the multiplex families were extracted. See FIG. 2a and FIG. 2b for an example of each of these two types of families. Three of the type I families and one of the type II families are from the same extended pedigree. However, the three type I families are only distantly related that they can be treated as independent for our purpose, and are included in our TDT analysis (yielding a total of 28 informative nuclear families). Among the 11 trios, there were 15 heterozygous parents with genotypes CD24^{226a/v}, of which 13 transmitted the v allele to their affected children. The contribution to the overall test statistic was thus X_{TDT}=13, much larger than the expected value of 7.5. Among the 17 sibships, the total number of v alleles among the affected siblings is X_{TDT}=20, still larger than the expected value of 18.57, although the discrepancy between the observed and the expected was not as striking as in the trios. Our Monte Carlo procedure with 1,000,000 simulated null data sets yielded a significant result for the combined test statistic, X_{obs}=X_{TDT}+X_{STDT}=33 (P=0.017). A pedigree TDT test that takes family dependency into account (31) yielded similarly significant result.

Taken together, both the TDT test for the family data and the Chi-square tests for the population data suggest that CD24^{v} allele is a significant risk factor for the incidence of MS.

CD24 Genotype Affects Progression of MS

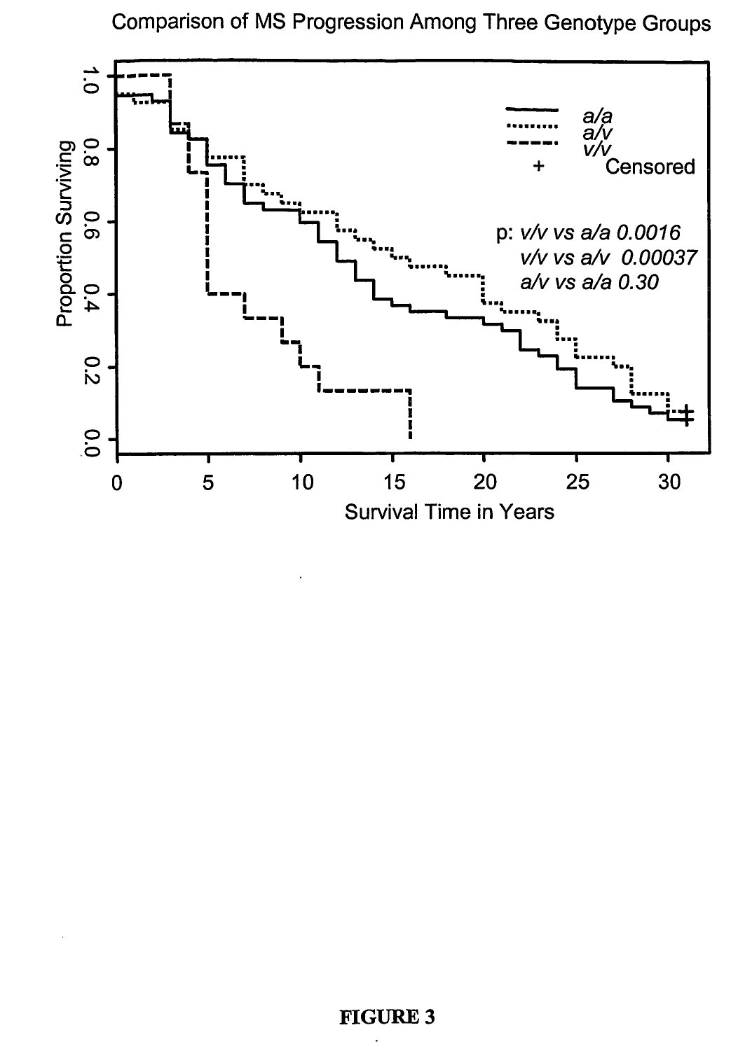

The MS disease severity is usually measured according to the expanded disability status scale (EDSS) score. MS patients that have lost the ability to walk without aid would have reached EDSS 6.0. For the majority of the patients, their EDSS 6.0 was based on follow-up at our center. A few of the cases were based on interview. Since this is one of the most traumatic events in the patient's life, most MS patients can recall accurately the time when their disease reached EDSS 6.0. We have chosen all patients that have EDSS of 6.0 or higher, which resulted in 57, 40, and 15 patients with genotype a/a, a/v, and v/v, respectively. We then tested whether the CD24 genotype affected the time span it took the patients to reach EDSS 6.0 from the day of the first symptom of MS. As shown in FIG. 3, 50% of the CD24^{226v/v} patients reached EDSS 6.0 in 5 years after the first symptom, whereas those with CD24^{226a/a} and CD24^{226a/v} genotypes reached EDSS 6.0 in 13 and 16 years, respectively.

Furthermore, comparison of the three estimated survival curves in FIG. 3 reveals that the CD24 genotypes have significant impact on the progression (p=0.0008). Pair-wise comparisons further show that CD24^{226v/v} patients progressed more rapidly towards EDSS 6.0 than both CD24^{226a/v} patients (p=0.00037) and CD24^{226a/a} patients (p=0.0016), even after Bonferroni correction. There is no significant difference between CD24^{226a/a} and CD24^{226a/v} patients (p=0.30).

Determination of Cell Surface Expression of CD24^{226v}

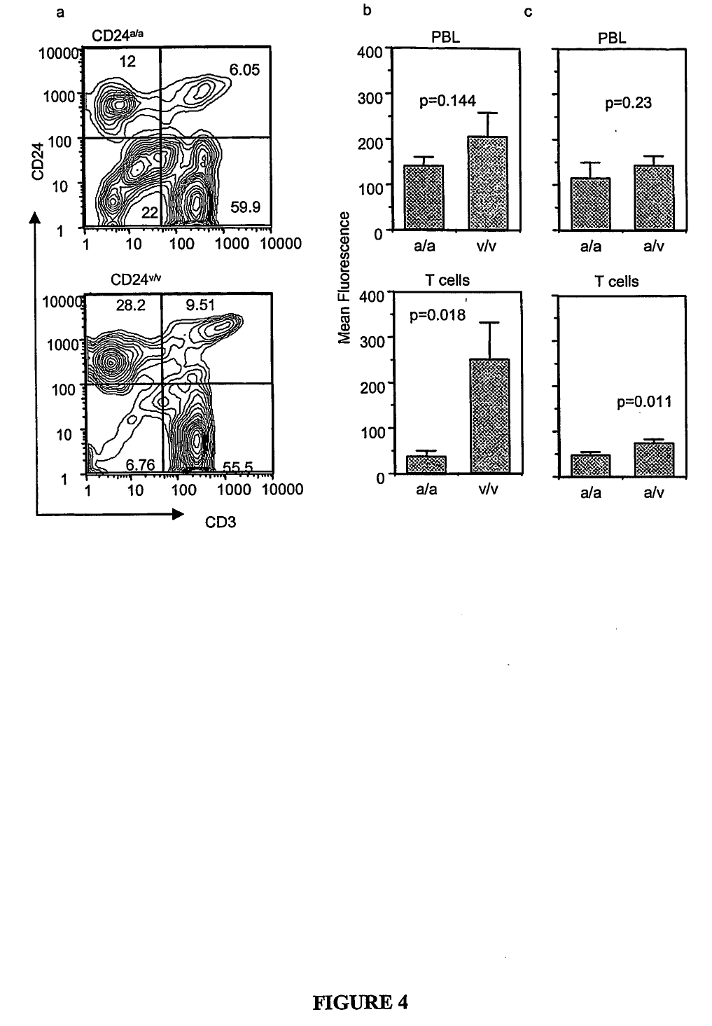

The CD24 is a GPI anchored molecule, and therefore needs to be cleaved of C-terminal sequence prior to GPI attachment (32, 33). This cleavage requires specific sequence at and near the cleavage site (ω), ω+1 and ω+2 sites (32, 33). Moreover, systematic analysis of all GPI anchored proteins with known cleavage sites suggests that although the amino acid at the ω-1 and ω-2 positions may have a quantitative effect on the cleavage efficiency, as the optimal cleavage requires that the side chains in the 4 positions have a combined volume of 430A³ (34). As shown in FIG. 1a, CD24^{226v} and CD24^{226a} have a non-conservative replacement of A by V at the ω-1 site. Since all 4 amino acids in CD24^{226a} have the small side chains (A and G), replacement of A with V at ω-1 may increase the efficiency of cleavage. As a result, the CD24^{226v} protein may be expressed at a higher level than the CD24^{226a} proteins. To test this notion, we analyzed CD24 expression on the peripheral blood leukocytes of age, sex and disease-status matched CD24^{226a/a} and CD24^{226v/v} MS patients (Table 1, experiment 1) by two-color flow cytometry. The profiles of a representative sample in each group were presented in FIG. 4a, while the mean fluorescence intensities of total PBL and CD3⁺ T cells among the PBL were summarized in FIG. 4b. As shown in FIG. 4a, CD24 is expressed on both T cells and non-T cells, regardless of the genotypes of the MS patients. However, the % of positive cells and intensity of expression were higher among the PBL of CD24^{226v/v} patients. Interestingly, CD3⁺ T cells from the CD24^{226a/a} patients expressed six-fold less cell-surface CD24 than those from the CD24^{226v/v} patients. While the same trend was found for total PBL, this was not statistically significant. In a separate experiment, we also compared 6 CD24^{226a/a} and 6 CD24^{226a/v} patients for the CD24 expression. Although the MS type was not well matched in this experiment, the MS type did not appear to influence the CD24 expression (Table 1). As shown in Table 1 (Exp. 2) and FIG. 4c, although the CD24^{226a/v} T cells expressed higher CD24 than the CD24^{226a/a} T cells, the increase is less than 2-fold. The small increase may explain why the CD24^{226a/v} genotype had no measurable effect on the risk and progression of MS.

TABLE 1
Profiles of patients and CD24 expression among MS patients with
different genotype

| | | | | | | Mean | |
|---|---|---|---|---|---|---|---|
| | | | | | | Fluorescence^{#} | |
| ID No. | Sex | Age | EDSS | CD24 | MS type* | PBL | T cells |
| Expt. 1 | | | | | | | |
| 8a | F | 60 | 7.0 | a/a | SP | 137 | 27 |
| 11z | M | 64 | 6.5 | a/a | SP | 85 | 34 |
| 15z | F | 24 | 2.0 | a/a | RR | 148 | 22 |
| 32a | F | 62 | 2.0 | a/a | RR | 201 | 29 |
| 76z | F | 57 | 6.5 | a/a | SP | 143 | 83 |
| 25a | F | 51 | 6.0 | v/v | RR | 225 | 210 |
| 27a | F | 50 | 2.0 | v/v | RR | 351 | 545 |
| 7y | F | 47 | 2.0 | v/v | RR | 58 | 51 |
| 118z | M | 70 | 7.0 | v/v | SP | 117 | 148 |
| 122z | F | 66 | 7.0 | v/v | SP | 283 | 302 |
| Expt. 2 | | | | | | | |
| 42z | F | 56 | 6.0 | a/a | SP | 71 | 35 |
| 43z | F | 43 | 2.0 | a/a | RR | 264 | 65 |
| 45z | F | 54 | 2.0 | a/a | RR | 56 | 20 |
| 46z | M | 61 | 7.5 | a/a | PP | 69 | 30 |
| 48z | M | 64 | 6.0 | a/a | PP | 180 | 66 |
| 12y | F | 59 | 6.5 | a/a | SP | 49 | 37 |
| 44z | F | 54 | 2.0 | a/v | RR | 204 | 92 |
| 47z | F | 33 | 2.0 | a/v | RR | 110 | 60 |
| 11y | F | 67 | 2.0 | a/v | RR | 158 | 52 |
| 21a | F | 51 | 5.0 | a/v | RR | 125 | 30 |
| 22a | M | 61 | 7.5 | a/v | SP | 185 | 92 |
| 23a | F | 59 | 2.5 | a/v | RR | 88 | 72 |

*The MS type are: RR, remitting relapsing; SP, secondary progressive; PP, primary progressive.
^{#}Samples from RR patients were collected during remitting phase.

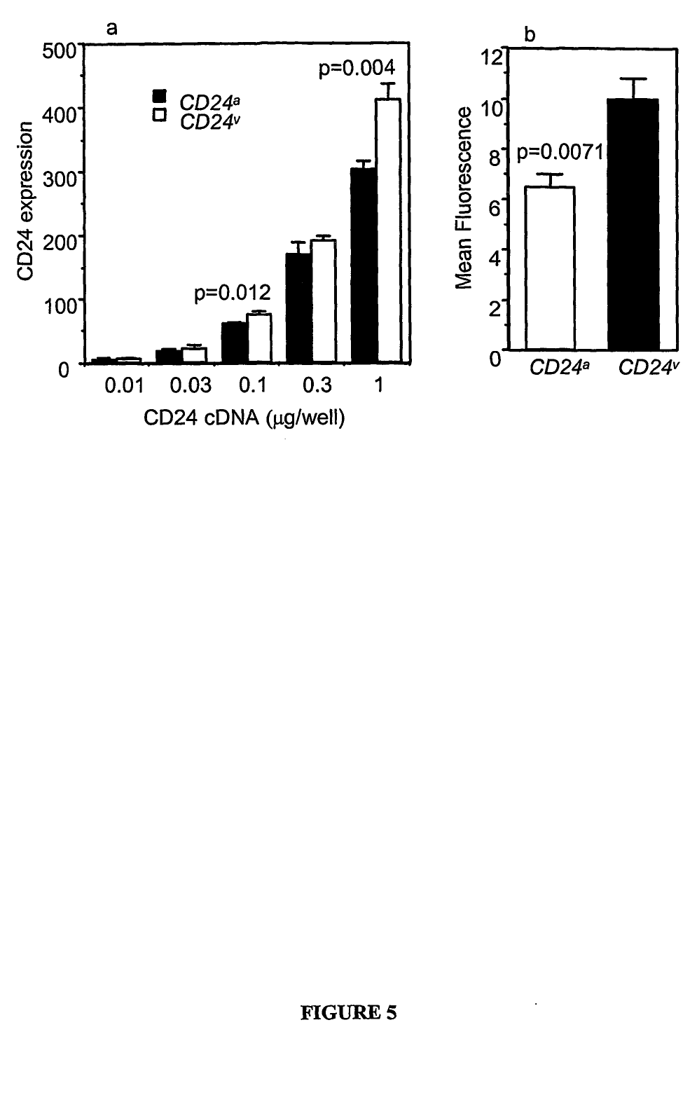

To directly address whether CD24 SNP caused variation in CD24 expression, we cloned both CD24^{226v} and CD24^{226a} cDNA and transfected the CHO cells with different concentrations of plasmids. Three days after the transfection, the cell surface expression of the CD24 gene was analyzed by flow cytometry. As shown in FIG. 5a, across a wide range of doses, the CD24^{226v} cDNA resulted in 30-40% more cell surface expression of CD24 when compared with the CD24^{226a} cDNA. To avoid variation in transfection, we also used the neomycin selection to remove untransfected cells, and compared the pooled drug resistant clones for their CD24 expression. Again, CD24^{226v} cDNA transfectants expressed significantly higher cell surface CD24 (FIG. 5b).

Isolation and SNP Genotype Analysis of Nucleic Acids

The genetic material to be assessed can be obtained from any nucleated cell from the individual being tested. For assay of genomic DNA, virtually any biological sample (other than pure red blood cells) is suitable. For example, convenient tissue samples include whole blood, semen, saliva, tears, urine, fecal material, sweat, skin and hair. For assay of cDNA or mRNA, the tissue sample must be obtained from cells in which the target nucleic acid is expressed, preferably from T lymphocytes.

The nucleotide which occupies the polymorphic site of interest (e.g., nucleotide position 226 in CD24) can be identified by a variety methods, such as Southern analysis of genomic DNA; direct mutation analysis by restriction enzyme digestion; Northern analysis of RNA; denaturing high pressure liquid chromatography (DHPLC); gene isolation and sequencing; hybridization of an allele-specific oligonucleotide with amplified gene products; single base extension (SBE); or analysis of the cell-surface expression of the CD24 protein. A sampling of suitable procedures is discussed below:

Allele-Specific Probes

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Hybridizations are usually performed under stringent conditions, for example, at a salt concentration of no more than 1 M and a temperature of at least 25° C. For example, conditions of 5.times.SSPE (750 mM NaCl, 50 mM NaPhosphate, 5 mM EDTA, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

Tiling Arrays

The polymorphisms can also be identified by hybridization to nucleic acid arrays, some examples of which are described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Allele-Specific Primers

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

Primers are selected within the conserved regions shown in the attached alignment 1 to amplify a fragment with proper size for optimal detection. One primer is located at each end of the sequence to be amplified. Such primers will normally be between 10 to 30 nucleotides in length and have a preferred length from between 18 to 22 nucleotides. The smallest sequence that can be amplified is approximately 50 nucleotides in length (e.g., a forward and reverse primer, both of 20 nucleotides in length, whose location in the sequences is separated by at least 10 nucleotides). Much longer sequences can be amplified. Preferably, the length of sequence amplified is between 75 and 250 nucleotides in length, and between 75 and 150 for Taqman assay.

One primer is called the “forward primer” and is located at the left end of the region to be amplified. The forward primer is identical in sequence to a region in the top strand of the DNA (when a double-stranded DNA is pictured using the convention where the top strand is shown with polarity in the 5′ to 3′ direction). The sequence of the forward primer is such that it hybridizes to the strand of the DNA which is complementary to the top strand of DNA.

The other primer is called the “reverse primer” and is located at the right end of the region to be amplified. The sequence of the reverse primer is such that it is complementary in sequence to, i.e., it is the reverse complement of a sequence in, a region in the top strand of the DNA. The reverse primer hybridizes to the top strand of the DNA.

PCR primers should also be chosen subject to a number of other conditions. PCR primers should be long enough (preferably 10 to 30 nucleotides in length) to minimize hybridization to greater than one region in the template. Primers with long runs of a single base should be avoided, if possible. Primers should preferably have a percent G+C content of between 40 and 60%. If possible, the percent G+C content of the 3′ end of the primer should be higher than the percent G+C content of the 5′ end of the primer. Primers should not contain sequences that can hybridize to another sequence within the primer (i.e., palindromes). Two primers used in the same PCR reaction should not be able to hybridize to one another. Although PCR primers are preferably chosen subject to the recommendations above, it is not necessary that the primers conform to these conditions. Other primers may work, but have a lower chance of yielding good results.

PCR primers that can be used to amplify DNA within a given sequence can be chosen using one of a number of computer programs that are available. Such programs choose primers that are optimum for amplification of a given sequence (i.e., such programs choose primers subject to the conditions stated above, plus other conditions that may maximize the functionality of PCR primers). One computer program is the Genetics Computer Group (GCG recently became Accelrys) analysis package which has a routine for selection of PCR primers. There are also several web sites that can be used to select optimal PCR primers to amplify an input sequence. One such web site is http://alces.med.umn.edu/rawprimer.html. Another such web site is http://www-genome.wi.mit.edu/cgi-bin/primer/primer3_www.cgi.

Direct-Sequencing

The direct analysis of the sequence of polymorphisms of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saild et al. (1986) Nature 324:163); Saiki et al (1989) Proc. Nati Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect CD24 alleles. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in To be et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Many of the methods described herein require amplification of DNA from target samples. This can be accomplished by e.g., PCR. See generally PCR Technology: Principles and Applications for DNA Amplification (ed. H. A. Erlich, Freeman Press, New York, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4, 560 (1989), Landegren et al., Science 241,1077 (1988), transcription amplification (Kwoh et al., Proc. Nati. Acad. Sci. USA 86,1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

Correlation of MS Phenotype with SNP Analyses

Correlation between a particular phenotype, e.g., MS symptoms, and the presence or absence of a particular CD24 SNP allele is performed for a population of individuals who have been tested for the presence or absence of the phenotype. Correlation can be performed by standard statistical methods such as a Chi-squared test and statistically significant correlations between polymorphic form(s) and phenotypic characteristics are noted. For example, as described herein, it has been found that the presence of the CD24 variant allele at nucleic acid position 226, with a replacement of the C at polymorphic site 226 with a T, correlates positively with MS with a p value of p=0.023 by Chi-squared test.

This correlation can be exploited in several ways. In the case of a strong correlation between a particular polymorphic form, detection of the polymorphic form in an individual may justify immediate administration of treatment, or at least the institution of regular monitoring of the individual. Detection of a polymorphic form correlated with a disorder in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic form and a particular disorder, immediate therapeutic intervention or monitoring may not be justified. Nevertheless, the individual can be motivated to begin simple life-style changes (e.g., diet modification, therapy or counseling) that can be accomplished at little cost to the individual but confer potential benefits in reducing the risk of conditions to which the individual may have increased susceptibility by virtue of the particular allele. Furthermore, identification of a polymorphic form correlated with enhanced receptiveness to one of several treatment regimes for a disorder indicates that this treatment regimen should be followed for the individual in question.

Furthermore, it may be possible to identify a physical linkage between a genetic locus associated with a trait of interest (e.g., MS) and polymorphic markers that are or are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (USA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage is analyzed by calculation of LOD (log of the odds) values. A LOD value is the relative likelihood of obtaining observed segregation data for a marker and a genetic locus when the two are located at a recombination fraction θ, versus the situation in which the two are not linked, and thus segregating independently (Thompson & Thompson, Genetics in Medicine (5th ed, W. B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (θ), ranging from θ=0.0 (coincident loci) to θ=0.50 (unlinked). Thus, the likelihood at a given value of θ is: probability of data if loci linked at θ to probability of data if loci unlinked. The computed likelihoods are usually expressed as the log₁₀ of this ratio (i.e., a LOD score). For example, a LOD score of 3 indicates 1000:1 odds against an apparent observed linkage being a coincidence. The use of logarithms allows data collected from different families to be combined by simple addition. Computer programs are available for the calculation of LOD scores for differing values of 6 (e.g., LIPED, MLINK (Lathrop, Proc. Nat. Acad. Sci. (USA) 81, 3443-3446 (1984)). For any particular LOD score, a recombination fraction may be determined from mathematical tables. See Smith et al., Mathematical tables for research workers in human genetics (Churchill, London, 1961); Smith, Ann. Hum. Genet. 32, 127-150 (1968). The value of theta. at which the LOD score is the highest is considered to be the best estimate of the recombination fraction.

Positive LOD score values suggest that the two loci are linked, whereas negative values suggest that linkage is less likely (at that value of theta.) than the possibility that the two loci are unlinked. By convention, a combined LOD score of +3 or greater (equivalent to greater than 1000:1 odds in favor of linkage) is considered definitive evidence that two loci are linked. Similarly, by convention, a negative LOD score of −2 or less is taken as definitive evidence against linkage of the two loci being compared. Negative linkage data are useful in excluding a chromosome or a segment thereof from consideration. The search focuses on the remaining non-excluded chromosomal locations.

### EXAMPLES

### Example 1

### PCR Amplification and RFLP Analysis of CD24 Gene

Collection of Samples

All sample collection and experimentation have been approved by the Institutional Review Board (IRB), and informed consents from all participants were obtained prior to sample collection. Patients with definite MS, as diagnosed by KR at the Ohio State University MS Center according to the McDonald criteria (25), were offered the opportunity to participate. Consenting family members with or without MS provided blood samples as well. When family members were in other sites, samples were obtained by a local physician or nurse and transported or mailed to our center. Ascertainment of presence or absence of MS amongst the relatives was by history only, and relatives who provided blood samples were not subject to neurological evaluation or Magnetic Resonance Imaging (MRI) at our center. Of the 498 samples that yielded valid genotyping information, 242 were from MS patients and 256 were from the non-MS relatives. Only multiplex families were used for association analysis.

The clinical diagnosis of MS type and the Expanded Disability Status Scale (EDSS) (26) were determined. The time of first onset and the time when the patients were first prescribed a walking aid (EDSS 6.0) was determined retrospectively by analysis of case record.

Leftover blood samples from American Red Cross at Columbus were used as population control. A total of 207 samples were selected on basis of availability only over a one-year period. It is therefore expected that the genetic distribution resembles that of the Central Ohio population from which most of the patients and their family members were recruited.

Analysis

The reported SNP for CD24 is a replacement of C at nucleotide (nt) 226 by T (C>T) in the coding region of exon 2 (Gene bank accession: NM_{—}013230), which results in a substitution of Ala at amino acid 57 by Val near the GPI-anchorage site of the mature protein. The genomic DNA was isolated from approximately 5×10⁶ human peripheral blood leukocytes (PBL) using QlAamp DNA blood mini-kit (Qiagen Inc, Valencia, Calif.). DNA fragments bearing this SNP site were amplified by PCR using a forward (ttg ttg cca ctt ggc att ttt gag gc) and a reverse primer (gga ttg ggt tta gaa gat ggg gaa a). The PCR conditions were: 94° C. for 1 min, 50° C. for 1 min and 72° C. for 1 min, for 35 cycles. The predicted CD24 PCR fragment is 453 bp long. The C>T change yielded a BstXI restriction enzyme site at nt 215, which allowed us to differentiate these two different CD24 alleles by RFLP analysis. Briefly, an aliquot of CD24 PCR products were digested with BstXI for 16 hours at 50° C. The digested products were then separated in a 2.5% agarose gel. The predicted digestion pattern is as follows: PCR products of T226 allele will be cut into two small fragments (317 bp and 136 bp), while those of the C226 will be completely resistant. A combination of the two types of the products at close to 50% levels will indicate the heterozygocity of the subject.

### Example 2

### Molecular Cloning and Expression of CD24^{a} and CD24^{v} cDNA

The CD24 cDNA was amplified from PBL or CD24^{v/v} and CD24^{a/a} individuals by RT-PCR. The primers used were: Forward (CD24F.H3): ggccaagcttatgggcagagcaatggtg; and reverse (CD24R.Xhol): atccctcgagttaagagtagagatgcag. The PCR products (256 bp) were digested with HindIII/Xhol and then cloned into pCDNA3 expression vector at HindIII/Xhol site, thus generating plasmid pCDNA3-CD24A and pCDNA3-CD24V. The sequence of CD24 cDNA inserts was confirmed by DNA sequencing. To test the expression efficiency of the two CD24 alleles, we transfected varying concentrations of the plasmids into the CHO cells using the fugene 6, as described (27). Three days after transfection, the cell surface expression of the CD24 was determined by flow cytometry, using saturating amounts of anti-CD24 antibodies.

### Example 3

### Evaluation of CD24^{a} and CD24^{v} Expression Using Flow Cytometry

Expression of human and mouse CD24 was determined by flow cytometry using fluorochrome-labeled anti-human (B-D Pharmingen, San Diego, Calif.). PBL were isolated from fresh blood samples and stained with saturating amounts of anti-CD24 antibodies in conjunction with anti-CD3 antibodies to mark the T cells among the PBL.

### Example 4

### Statistical Analysis

Case-Control Population Study

MS patients and normal controls were examined for significant differences in their genotype distributions in the CD24 SNP at the population level. Most of the cases and the control subjects were from Central Ohio, reflecting, at least to some extent, a similarity in the disease and control populations. Pearson's Chi-square test (28) was used to perform the homogeneity test between the two distributions of the genotypes. In addition, we performed further tests to compare the frequencies of CD24^{v/v} genotype between the cases and controls, again using the Chi-square tests, but with Yates' correction. Since the number of individuals falling into each of the three genotypes in both the cases and controls is fairly large, the Chi-square tests should yield valid estimates of the p-values.

Association Test for Transmission Disequilibrium of the V Allele.

Since results from population studies can be affected by population admixture and stratification, we also carried out transmission disequilibrium test (TDT) using family data. Families with at least two MS patients (multiplex families) are ascertained for our genetic analysis to determine whether, in families that exhibit evidence of familial aggregation, the v allele in the CD24 SNP is transmitted preferentially to MS patients.

Two types of informative nuclear families were extracted from the multiplex families and included in our analysis. The type I families (trios) are those in which there is one MS patient and both parental genotypes are available with at least one being heterozygous. The type II families (sibships) are those in which both affected and unaffected siblings are available with at least two different genotypes in the sibship. For a family that can be of either type I or type II, it is classified to be a type I family following the recommendation of Spielman and Ewens (29).

A combined TDT (for type I families) and STDT (for type II families) test, as suggested by Spielman and Ewens (29), but with a Monte Carlo procedure for estimating the p-value, is employed. Specifically, let X_{TDT} denote the total number of V alleles transmitted to the MS patients from heterozygous parents in the type I families. Let X_{STDT} denote the total number of V alleles among the affected siblings in the type II families. Then X_{obs}=X_{TDT}+X_{STDT} is the observed test statistic for all informative families combined. Although one could estimate the p-value using normal asymptotic as suggested in Spielman and Ewens (29), we opted for the Monte Carlo procedure described in the following to avoid the need to rely on an asymptotic distribution with a moderate sample size.

To estimate the p-value of the test, 1,000,000 replicated datasets, under the null hypothesis that the CD24 SNP is unlinked to an MS locus, are generated as follows. For each type I family, we randomly select one of the two alleles in each parent to make up the new genotype of the patient, while the parental genotypes are unchanged. For each type II family, we follow the scheme of Spielman and Ewens (29) by simply permuting the affection status of the individuals in the sibship. For each simulated replicate, a test statistic X is computed. The p-value is taken to be the proportion of the Xs that are equal to, or greater than, the observed statistic, X_{obs}, in the actual data. This Monte Carlo estimate of the p-value should be very close to the true p-value given the large number of replicates performed.

Comparison of Survival Curves.

Patients with MS severity reaching EDSS 6.0 or higher are classified into three groups according to their CD24 genotypes. To assess whether MS progression is different among patients with different genotypes, we first estimated the survival curve, using the Kaplan-Meier method, for each of the three groups, two of which having right censored data. Then the estimated Kaplan-Meier survival curves are compared using the log-rank test (30). Here, survival is taken to mean that a patient has not reached EDSS 6.0 yet, and the time span is measured by the number of years lapsed since the first symptom.

### Example 5

### Analysis of Additional Polymorphisms in the 3′ Untranslated Region (UTR) of CD24 mRNA

The CD24 gene was amplified from eight (8) randomly selected normal individuals from Columbus Red Cross donor samples using primers that cover the ends of intron 1 and exon 2. Forty-four clones were sequenced and compared for the polymorphism within the exon 2 sequence. To avoid errors, only those replacements found in more than one independent clone were considered. The data are summarized in Table 2, below.

TABLE 2
CD24 alleles identified from 8 individuals.

Polymorphism

| | | | | | | | |
|---|---|---|---|---|---|---|---|
| ID | Clones | 226C/T | 475A/G | 1110A/G | 1580--/TG | 1678A/G | Allotypes (N)* |

| | | | | | | | |
|---|---|---|---|---|---|---|---|
| 1 | 8 | C(Ala) | G | G | — | G | a (3) |
| | | C(Ala) | A | A | TG | A | b (5) |
| 2 | 8 | C(Ala) | A | G | TG | A | c (4) |
| | | C(AIa) | A | A | TG | A | b (4) |
| 3 | 8 | C(Ala) | A | G | — | G | d (6) |
| | | C(Ala) | A | G | TG | A | c (2) |
| 4 | 5 | T(Val) | A | G | TG | G | e (5) |
| 5 | 2 | C(Ala) | A | G | — | G | d (2) |
| 6 | 3 | C(Ala) | A | A | TG | A | b (3) |
| 7 | 5 | C(Ala) | A | A | TG | A | b (5) |
| 8 | 5 | C(Ala) | A | G | TG | A | c (5) |

Five different allotypes, a-e were identified, N, number of sequence from given individual with that genotype. Together, a is confirmed by 3 individual clones; b, 17 clones; c, 11 clones: d, 8 clones: e, 5 clones.

Several conclusions can be made from the data summarized in Table 2. First, the CD24 loci can be extremely polymorphic, as five different SNPs have been identified in eight individuals. Second, at least four allotypes were identified within the previously classified CD24^{a} individuals. This will make a large number of previously un-informative families useful for the proposed studies, thus substantially improving the power of the analysis.

### Example 6

### CD24 Polymorphism at 3′ UTR and Risk of MS

We carried out an extensive analysis of the SNPs in our collection of case and control samples. Since 475A/G was not observed in any of the additional samples tested, we have focused our analysis on SNPs at 4 positions 226C/T, 1110A/G, 1580—/TG and 1678A/G.

We have analyzed 241 control and 221 case samples for the 4 polymorphic sites. Our analyses revealed that, in addition to previously identified 226C/T, 1110A/G polymorphism also showed significant association with risk of MS (P<0.01).

To analyze different alleles of CD24 genes are preferentially transmitted to MS patients, we tested samples collected from 101 families for their polymorphism in position 226, 1110, 1580 and 1678. As shown in Table 2, using three different programs (Refs 1-3), we have uncovered the strongest association between 1110G allele with MS. The significance of other SNP requires further testing.

TABLE 3
Summary data from family collected from Ohio

| SNP | | | |
|---|---|---|---|
| (Associated | PDT | FBAT | TRANSMIT |
| alleles) | (fam, Trio, DSP) | (101/92/406^{a}) | (88^{b}) |
| 6 (T) | 0.243 (76, 39, 120) | 0.569 | 0.733 |
| 1110 (G) | 0.0142 (71, 31, 114) | 0.0288 | 0.043 |
| 1580(*) | 0.128 (71, 31, 114) | 0.170 | 0.009 |
| 1678(*) | 0.829 (71, 31, 115) | 0.786 | 0.881 |

^{a}101 pedigree, 92 nuclear families, 406 persons
^{b}No. Families with transmission to affected offspring
(*)Different program indicate different allele is involved.

When we extended the number of multiplex families, we were able to confirm our previous studies that 226(T) allele associate with MS. Again, 1110(G) has the strongest association with MS, regardless of the statistical methods.

TABLE 4
Summary data from multiplex family collected from Ohio

| SNP | PDT | FBAT | |
|---|---|---|---|
| (Associated alleles) | (fam, Trio, DSP) | (53/52/240^{a}) | TRANSMIT |
| 226 (T) | 0.038 (39, 16, 85) | 0.135 | 0.222 (47) |
| 1110 (G) | 0.028 (37, 12, 79) | 0.021 | 0.034 (48) |
| 1580(*) | 0.423 (37, 12, 79) | 0.604 | 0.131 (48) |
| 1678(*) | 0.529 (37, 12, 79) | 0.477 | 0.799 (48) |

### Example 7

### Polymorphism at Position 1580 and MS Progression

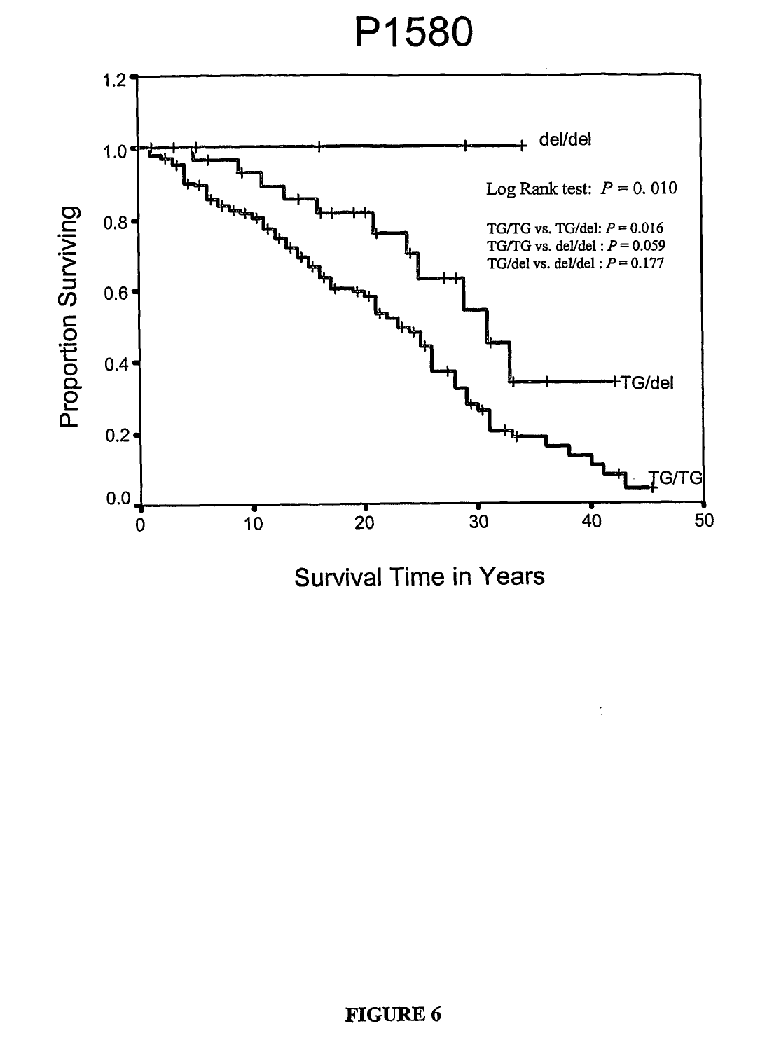

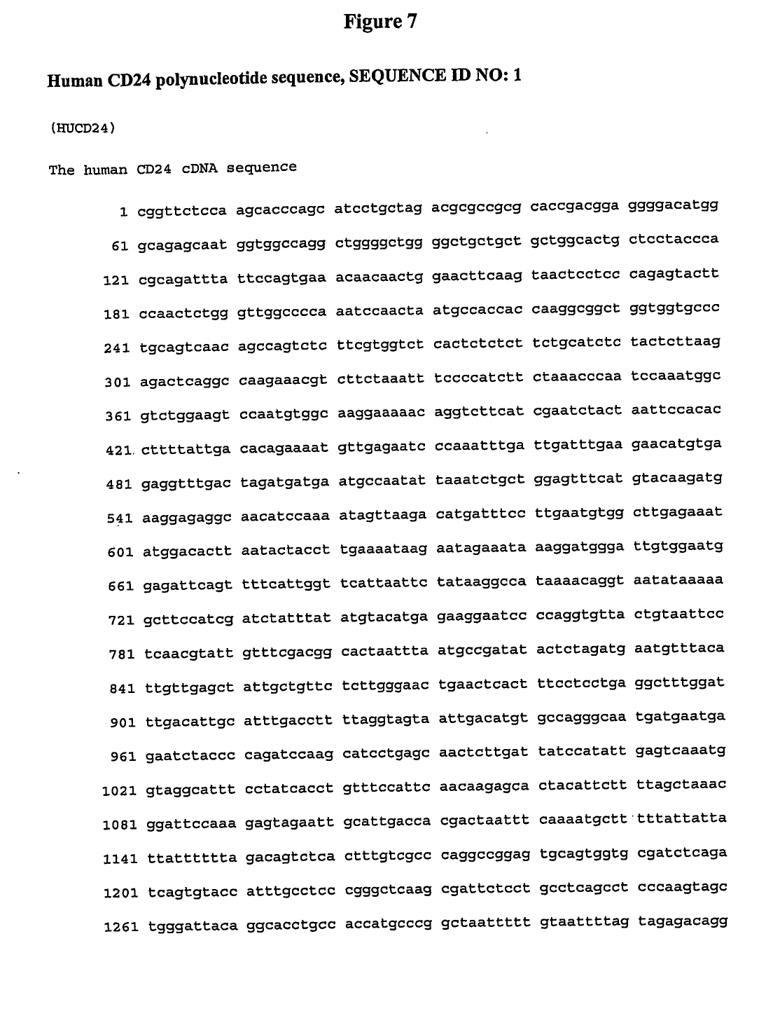

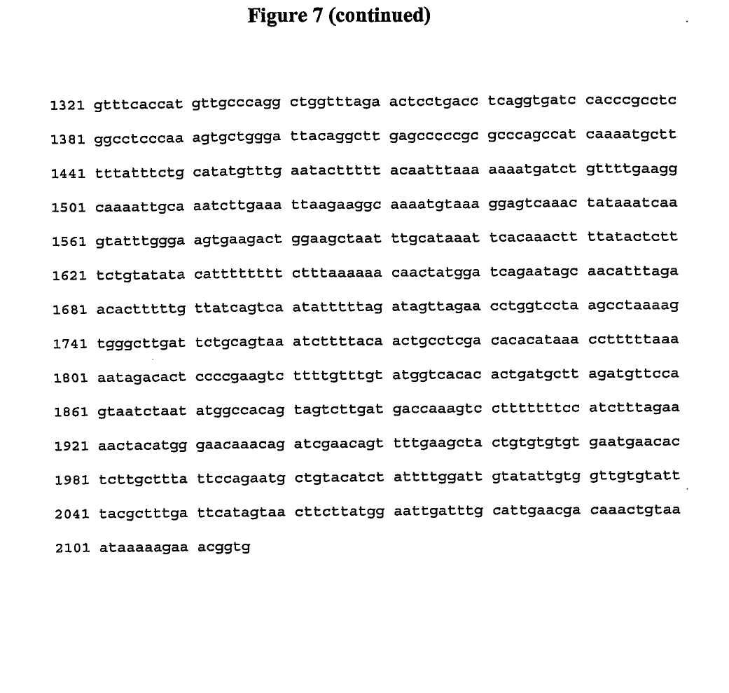

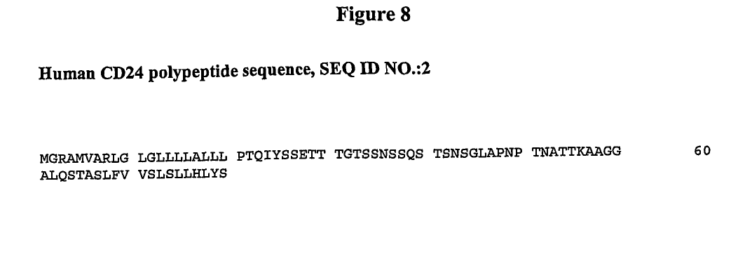

Survival analysis revealed that SNP at 1580 have significant impact for the progression of MS. As shown in FIG. 6, the genotypes at this position associate with the time span from the day of first MS-like symptom to the day when the patients requires walking aid.

Other embodiments of the invention will be apparent to those skilled in the art from consideration of the specification and practice of the invention disclosed herein. It is intended that the specification and examples be considered as exemplary only, with a true scope and spirit of the invention being indicated by the appended claims.

### REFERENCES

1. Noseworthy, J. H. (1999) Nature 399, A40-7.
2. Carton, H., Vlietinck, R., Debruyne, J., De Keyser, J., D'Hooghe, M. B., Loos, R., Medaer, R., Truyen, L., Yee, I. M. & Sadovnick, A. D. (1997) J Neurol Neurosurg Psychiatry 62, 329-33.
3. Ebers, G. C., Bulman, D. E., Sadovnick, A. D., Paty, D. W., Warren, S., Hader, W., Murray, T. J., Seland, T. P., Duquette, P., Grey, T. & et al. (1986) N EngI J Med 315, 1638-42.
4. Kellar-Wood, H. F., Wood, N. W., Holmans, P., Clayton, D., Robertson, N. & Compston, D. A. (1995) J Neuroimmunol 58, 183-90.
5. Miller, D. H., Hornabrook, R. W., Dagger, J. & Fong, R. (1989) J Neurol Neurosurg Psychiatry 52, 575-7.
6. Morling, N., Sandberg-Wollheim, M., Fugger, L., Georgsen, J., Hylding-Nielsen, J. J., Madsen, H. O., Rieneck, K., Ryder, L. & Svejgaard, A. (1992) Immunogenetics 35, 391-4.
7. Olerup, 0. & Hillert, J. (1991) Tissue Antigens 38, 1-15.
8. Haines, J. L., Ter-Minassian, M., Bazyk, A., Gusella, J. F., Kim, D. J., Terwedow, H., Pericak-Vance, M. A., Rimmier, J. B., Haynes, C. S., Roses, A. D., Lee, A., Shaner, B., Menold, M., Seboun, E., Fitoussi, R. P., Gartioux, C., Reyes, C., Ribierre, F., Gyapay, G., Weissenbach, J., Hauser, S. L., Goodkin, D. E., Lincoln, R., Usuku, K., Oksenberg, J. R. & et al. (1996) Nat Genet 13, 469-71.
9. Sawcer, S., Jones, H. B., Feakes, R., Gray, J., Smaldon, N., Chataway, J., Robertson, N., Clayton, D., Goodfellow, P. N. & Compston, A. (1996) Nat Genet 13, 464-8.
10. Ebers, G. C., Kukay, K., Bulman, D. E., Sadovnick, A. D., Rice, G., Anderson, C., Armstrong, H., Cousin, K., Bell, R. B., Hader, W., Paty, D. W., Hashimoto, S., Oger, J., Duquette, P., Warren, S., Gray, T., O'Connor, P., Nath, A., Auty, A., Metz, L., Francis, G., Paulseth, J. E., Murray, T. J., Pryse-Phillips, W., Risch, N. & et al. (1996) Nat Genet 13, 472-6.
11. Schmidt, S., Barcellos, L. F., DeSombre, K., Rimmler, J. B., Lincoln, R. R., Bucher, P., Saunders, A. M., Lai, E., Martin, E. R., Vance, J. M., Oksenberg, J. R., Hauser, S. L., Pericak-Vance, M. A. & Haines, J. L. (2002) Am J Hum Genet 70, 708-17.
12. Kuokkanen, S., Sundvall, M., Terwilliger, J. D., Tienari, P. J., Wikstrom, J., Holmdahl, R., Peftersson, U. & Peltonen, L. (1996) Nat Genet 13, 477-80.
13. Bai, X. F., Liu, J. Q., Liu, X., Guo, Y., Cox, K., Wen, J., Zheng, P. & Liu, Y. (2000) J Clin Invest 105, 1227-32.
14. Hubbe, M. & Altevogt, P. (1994) Eur J Immunol 24, 731-7.
15. Zhou, Q., Wu, Y., Nielsen, P. J. & Liu, Y. (1997) Eur J Immunol 27, 2524-8.
16. Liu, Y., Jones, B., Aruffo, A., Sullivan, K. M., Linsley, P. S. & Janeway, C. A., Jr. (1992) J Exp Med 175, 43745.
17. De Bruijn, M. L., Peterson, P. A. & Jackson, M. R. (1996) J Immunol 156, 2686-92.
18. Enk, A. H. & Katz, S. I. (1994) J Immunol 152, 3264-70.
19. Liu, Y., Jones, B., Brady, W., Janeway, C. A., Jr., Linsley, P. S. & Linley, P. S. (1992) Eur J Immunol 22, 2855-9.
20. Liu, Y., Wenger, R. H., Zhao, M. & Nielsen, P. J. (1997) J Exp Med 185, 251-62.
21. Wu, Y., Zhou, Q., Zheng, P. & Liu, Y. (1998) J Exp Med 187, 1151-6.
22. Hahne, M., Wenger, R. H., Vestweber, D. & Nielsen, P. J. (1994) J Exp Med 179, 1391-5.
23. Baron, J. L., Madri, J. A., Ruddle, N. H., Hashim, G. & Janeway, C. A., Jr. (1993) J Exp Med 177, 57-68.
24. Zarn, J. A., Jackson, D. G., Bell, M. V., Jones, T., Weber, E., Sheer, D., Waibel, R. & Stahel, R. A. (1995) Cytogenet Cell Genet 70, 119-25.
25. McDonald, W. I., Compston, A., Edan, G., Goodkin, D., Hartung, H. P., Lublin, F. D., McFarland, H. F., Paty, D. W., Polman, C. H., Reingold, S. C., Sandberg-Wollheim, M., Sibley, W., Thompson, A., van den Noort, S., Weinshenker, B. Y. & Wolinsky, J. S. (2001) Ann Neurol 50, 121-7.
26. Kurtzke, J. F. (1983) Neurology 33, 1444-52.
27. Liu, X., Bai, X. F., Wen, J., Gao, J.-X., Liu, J., Lu, P., Wang, Y., Zheng, P. & Liu, Y. (2001) J. Exp. Med. 194, 1339-1348.
28. Agresti, A. (1990) New York: John Weiley & Sons.
29. Spielman, R. S. & Ewens, W. J. (1998) Am J Hum Genet 62, 450-8.
30. Fleming, T. R. & Harrington, D. P. (1991) Counting processes & survival analysis (John Wiley and Sons., New York).
31. Martin, E. R., Monks, S. A., Warren, L. L. & Kaplan, N. L. (2000) Am J Hum Genet 67, 146-54.
32. Englund, P. T. (1993) Annu Rev Biochem 62, 121-38.
33. Udenfriend, S. & Kodukula, K. (1995) Annu Rev Biochem 64, 563-91.
34. Eisenhaber, B., Bork, P. & Eisenhaber, F. (1998) Protein Eng 11, 1155-61.
35. Haines, J. L., Terwedow, H. A., Burgess, K., Pericak-Vance, M. A., Rimmler, J. B., Martin, E. R., Oksenberg, J. R., Lincoln, R., Zhang, D. Y., Banatao, D. R., Gatto, N., Goodkin, D. E. & Hauser, S. L. (1998) Hum Mol Genet 7, 1229-34.

What is claimed is:

1. A method for predicting the likelihood that an individual will develop multiple sclerosis, comprising:
a) obtaining a nucleic acid sample from an individual to be assessed; and
b) determining the nucleotide present at the nucleotide position corresponding to position 226 of the native CD24 gene in the individual which sequence corresponds to SEQ ID NO: 1,
wherein the presence of an thymidine at position 226 indicates that the individual has a greater likelihood of being diagnosed with multiple sclerosis than an individual having a cytosine at that position.

2. A method for predicting the likelihood that an individual will develop multiple sclerosis, comprising:
a) obtaining a nucleic acid sample from an individual to be assessed; and
b) determining the nucleotide present at the nucleotide position corresponding to position 1110 of the native CD24 gene in the individual which sequence corresponds to SEQ ID NO: 1,
wherein the presence of a guanine at position 1110 indicates that the individual has a greater likelihood of being diagnosed with multiple sclerosis than an individual having an adenine at that position.

3. The method according to either of claims 1 or 2, wherein the individual is an individual at risk for development multiple sclerosis based on the presence of an allelic variant of HLA.

4. The method according to either of claims 1 or 2, wherein the individual exhibits clinical symptoms of multiple sclerosis.

5. The method according to either of claims 1 or 2, wherein at least one blood relative of the individual has been diagnosed with multiple sclerosis.

6. A method for predicting the likelihood that an individual who has been diagnosed with multiple sclerosis will experience rapid progression of multiple sclerosis, comprising:
a) obtaining a nucleic acid sample from an individual to be assessed; and
b) determining the nucleotide present at the nucleotide position corresponding to position 226 of the native CD24 gene in the individual which sequence corresponds to SEQ ID NO: 1,
wherein the presence of an thymidine at position 226 indicates that the individual has a greater likelihood of experiencing rapid progression of multiple sclerosis than an individual diagnosed with multiple sclerosis and having an cytosine at that position.

7. A method for predicting the likelihood that an individual who has been diagnosed with multiple sclerosis will experience rapid progression of multiple sclerosis, comprising:
a) obtaining a nucleic acid sample from an individual to be assessed; and
b) determining if there is a deletion at positions 1580 and 1581 of the native CD24 gene in the individual, which sequence corresponds to SEQ ID NO: 1,
wherein deletions of TG at positions 1580 and 1581 indicate that the individual has a greater likelihood of experiencing rapid progression of multiple sclerosis than an individual diagnosed with multiple sclerosis and having TG at those positions.

8. A method of diagnosing or aiding in the diagnosis of multiple sclerosis in an individual comprising:
a) obtaining a nucleic acid sample from the individual;
b) determining the HLA genotype of the individual; and
c) determining the nucleotide present at nucleotide position 226 of the CD24 gene, wherein the presence of the HLA-DR2 genotype together with the presence of a thymidine at position 226 of the CD24 gene is indicative that the individual is more likely to develop multiple sclerosis as compared with an individual lacking the HLA-DR2 genotype and having a cytosine at position 226 of the CD24 gene.

9. A method of diagnosing or aiding in the diagnosis of multiple sclerosis in an individual comprising:
a) obtaining a nucleic acid sample from the individual;
b) determining the HLA genotype of the individual; and
c) determining the nucleotide present at nucleotide position 1110 of the CD24 gene,
wherein the presence of the HLA-DR2 genotype together with the presence of a guanine at position 1110 of the CD24 gene is indicative that the individual is more likely to develop multiple sclerosis as compared with an individual lacking the HLA-DR2 genotype and having an adenine at position 1110 of the CD24 gene.

10. A method for predicting the likelihood that an individual will develop multiple sclerosis, comprising:
a) obtaining a cell sample from an individual to be assessed;
b) determining the level of cell surface expression of CD24 protein on the surface of said cells; and
c) determining a base-line level of cell surface expression of the CD24 protein on control cells,
wherein an increased level of expression of CD24 on the cells isolated from the individual as compared with the control cells indicates that the individual has a thymidine at position 226 of the CD24 gene, and therefore has a greater likelihood of being diagnosed with multiple sclerosis than an individual having a cytosine at that position.

11. A method for predicting the likelihood that an individual will develop multiple sclerosis, comprising:
a) obtaining a cell sample from an individual to be assessed;
b) determining the level of cell surface expression of CD24 protein on the surface of said cells; and
c) determining a base-line level of cell surface expression of the CD24 protein on control cells,
wherein an increased level of expression of CD24 on the cells isolated from the individual as compared with the control cells indicates that the individual has a guanine at position 1110 of the CD24 gene, and therefore has a greater likelihood of being diagnosed with multiple sclerosis than an individual having a adenine at that position.

12. The method according to either of claims 10 or 11, wherein the cell sample comprises peripheral blood lymphocytes.

13. The method according to either of claims 10 or 11, wherein the cell sample comprises T lymphocytes.

14. The method according to either of claims 10 or 11, wherein the individual is an individual at risk for development multiple sclerosis based on the presence of an allelic variant of HLA.

15. The method according to either of claims 10 or 11, wherein the individual exhibits clinical symptoms of multiple sclerosis.

16. The method according to either of claims 10 or 11, wherein at least one blood relative of the individual has been diagnosed with multiple sclerosis.

17. A method for predicting the likelihood that an individual will develop multiple sclerosis, comprising:
a) obtaining a nucleic acid sample from an individual to be assessed;
b) screening the entire nucleotide sequence encoding the human CD24; and
c) detecting the presence of one or more polymorphisms of the CD24,
wherein the presence of an thymidine at position 226, and the presence of at least one other variant allele in the polynucleotide encoding CD24 that has been shown to have a positive correlation with increased risk for developing MS based on both population study and on transmission disequilibrium analysis, indicates that the individual has a greater likelihood of developing multiple sclerosis than an individual having a cytosine at position 226 and lacking any other variant alleles in the polynucleotide encoding CD24 that has been shown to have a positive correlation with increased risk for developing MS based on both population study and on transmission disequilibrium analysis.

18. A method for predicting the likelihood that an individual will develop multiple sclerosis, comprising:
a) obtaining a nucleic acid sample from an individual to be assessed;
b) screening the entire nucleotide sequence encoding the human CD24; and
c) detecting the presence of one or more polymorphisms of the CD24,
wherein the presence of an guanine at position 1110, and the presence of at least one other variant allele in the polynucleotide encoding CD24 that has been shown to have a positive correlation with increased risk for developing MS based on both population study and on transmission disequilibrium analysis, indicates that the individual has a greater likelihood of developing multiple sclerosis than an individual having an adenine at position 1110 and lacking any other variant alleles in the polynucleotide encoding CD24 that has been shown to have a positive correlation with increased risk for developing MS based on both population study and on transmission disequilibrium analysis.
